(19) United States
(12) Patent Application Publication
(10) Pub. No.: US 2024/0050708 A1
(43) Pub. Date: Feb. 15, 2024

(54) Optical Tip-Tracking Systems and Methods Thereof

(71) Applicant: Bard Access Systems, Inc., Salt Lake City, UT (US)

(72) Inventor: Anthony K. Misener, Bountiful, UT (US)

(21) Appl. No.: 18/383,809

(22) Filed: Oct. 25, 2023

Related U.S. Application Data
(63) Continuation of application No. 17/105,310, filed on Nov. 25, 2020, now Pat. No. 11,850,338.
(60) Provisional application No. 62/940,107, filed on Nov. 25, 2019.

(51) Int. Cl. A61M 25/09; A61B 34/20

(52) U.S. Cl. CPC A61M 25/09; A61B 34/20; A61M 2025/09175; A61B 2034/2055

(57) ABSTRACT

An optical tip-tracking system is disclosed including a light-emitting stylet, a light detector, and a console configured to operably connect to the light-emitting stylet and the light detector. The light-emitting stylet can be disposed in a lumen of a catheter. The light-emitting stylet can include a light source in a distal-end portion of the light-emitting stylet configured to emit light. The light detector can be placed over a patient. The light detector can include a plurality of photodetectors configured to detect the light emitted from the light source. The console can be configured to instantiate an optical tip-tracking process for optically tracking the distal-end portion of the light-emitting stylet while the light-emitting stylet is disposed in a vasculature of the patient, the light source is emitting light, the light detector is disposed over the light-emitting stylet, and the photodetectors are detecting the light emitted from the light source.

### PRIORITY

This application is a continuation of U.S. patent application Ser. No. 17/105,310, filed Nov. 25, 2020, which claims the benefit of priority to U.S. Patent Application No. 62/940,107 filed Nov. 25, 2019, each of which is incorporated by reference in its entirety into this application.

### BACKGROUND

Intravascular guidance of medical devices such as guidewires and catheters typically requires fluoroscopic methods for tracking tips of the medical devices and determining whether the tips are appropriately localized in their target anatomical structures. Such fluoroscopic methods expose clinicians and patients alike to harmful X-ray radiation. In some cases, the patients are exposed to potentially harmful contrast media needed for the fluoroscopic methods. Magnetic and electromagnetic means for tracking the tips of the medical devices obviate some of the foregoing issues with respect to exposure to radiation and potentially harmful contrast media, but the magnetic and electromagnetic means for tracking the tips of the medical devices are prone to interference.

Disclosed herein are optical tip-tracking systems and methods thereof that address the foregoing.

### SUMMARY

Disclosed herein is an optical tip-tracking system including, in some embodiments, a light-emitting stylet, a light detector, and a console configured to operably connect to the light-emitting stylet and the light detector. The light-emitting stylet is configured to be disposed in a lumen of a catheter. The light-emitting stylet includes a light source in a distal-end portion of the light-emitting stylet configured to emit light. The light detector is configured to be placed over a patient. The light detector includes a plurality of photodetectors configured to detect the light emitted from the light source. The console includes memory and a processor configured to instantiate an optical tip-tracking process for optically tracking the distal-end portion of the light-emitting stylet while the light-emitting stylet is disposed in a vasculature of the patient, the light source is emitting light, the light detector is disposed over the light-emitting stylet, and the photodetectors are detecting the light emitted from the light source.

In some embodiments, the light source is a light-emitting diode (“LED”).

In some embodiments, the light emitted from the light source has a center wavelength between about 650 nm to 1350 nm.

In some embodiments, the light emitted from the light source has a center wavelength between about 650 nm to 950 nm.

In some embodiments, the distal-end portion of the light-emitting stylet is configured to directionally emit light in one or more chosen directions.

In some embodiments, the light detector includes a housing having a patient-facing portion of the housing configured to transmit at least a portion of the light emitted from the light source to the photodetectors.

In some embodiments, the housing has a light-blocking portion of the housing opposite the patient-facing portion configured to block ambient light from the photodetectors.

In some embodiments, the photodetectors are arranged in an array such that the light emitted from the light source remains detectable by at least one photodetector of the photodetectors even when the light emitted from the light source is anatomically blocked from another one or more photodetectors of the photodetectors.

In some embodiments, the optical tip-tracking process is configured to provide tracking information as input to a display server of the console for optically tracking the distal-end portion of the light-emitting stylet in a graphical user interface on a display.

In some embodiments, the light-emitting stylet is configured to directly connect to the console.

In some embodiments, the light-emitting stylet is configured to indirectly connect to the console through an intervening multi-use cable.

In some embodiments, the light-emitting stylet is configured to indirectly connect to the console through the light detector.

In some embodiments, the light detector is configured to be placed over the patient and under a sterile drape. The light-emitting stylet includes a drape-piercing connector having a piercing element configured to pierce the sterile drape and insert into a receptacle of a light-detector connector extending from the light detector under the drape.

Also disclosed herein is an optical tip-tracking system including, in some embodiments, a catheter, a light-emitting stylet for the catheter, a light detector, and a console configured to operably connect to the light-emitting stylet and the light detector. The catheter includes a lumen extending through the catheter. The light-emitting stylet is configured to be disposed in a lumen of a catheter. The light-emitting stylet includes a light-emitting diode (“LED”) in a distal-end portion of the light-emitting stylet configured to emit light having a center wavelength between about 650 nm to 1350 nm. The light detector is configured to be placed over a patient. The light detector includes a plurality of photodetectors configured to detect the light emitted from the LED. The console includes memory and a processor configured to instantiate an optical tip-tracking process for optically tracking the distal-end portion of the light-emitting stylet in a graphical user interface on a display while the light-emitting stylet is disposed in a vasculature of the patient, the LED is emitting light, the light detector is disposed over the light-emitting stylet, and the photodetectors are detecting the light emitted from the LED.

In some embodiments, the light detector is configured to be placed over the patient and under a sterile drape. The light-emitting stylet includes a drape-piercing connector configured to pierce the sterile drape and connect with a light-detector connector extending from the light detector under the drape.

Also disclosed herein is an optical tip-tracking system including, in some embodiments, a light-emitting stylet, a light detector, and a console configured to operably connect to the light-emitting stylet and the light detector. The light-emitting stylet is configured to be disposed in a lumen of a catheter. The light-emitting stylet includes an optical fiber configured to convey light to a distal-end portion of the light-emitting stylet for emitting light therefrom. The light detector is configured to be placed over a patient. The light detector includes a plurality of photodetectors configured to detect the light emitted from the light source. The console includes a light source for the light emitting stylet and memory and a processor. The memory and the processor are configured to instantiate an optical tip-tracking process for optically tracking the distal-end portion of the light-emitting stylet while the light-emitting stylet is disposed in a vasculature of the patient, the light source is emitting light, the light detector is disposed over the light-emitting stylet, and the photodetectors are detecting the light emitted from the light source.

Also disclosed herein is a method of an optical tip-tracking system including, in some embodiments, a disposing step of disposing a light-emitting stylet of the optical tip-tracking system in a lumen of a catheter. The light-emitting stylet includes a light source in a distal-end portion of the light-emitting stylet. The method also includes a placing step of placing a light detector of the optical tip-tracking system over a patient. The light detector includes a plurality of photodetectors. The method also includes an advancing step of advancing the catheter from an insertion site to a destination within a vasculature of the patient while emitting light from the light source and detecting the light with the photodetectors. The method also includes a viewing step of viewing a display screen of the optical tip-tracking system while the display screen graphically tracks the distal-end portion of the light-emitting stylet through the vasculature of the patient.

In some embodiments, the light source of the light-emitting stylet distally extends beyond a distal end of the catheter while advancing the catheter, thereby enabling the photodetectors of the light detector to detect the light emitted from the light source.

In some embodiments, the method also includes a placing step of placing a sterile drape over both the patient and the light detector. The method also includes a connecting step of connecting a drape-piercing connector of the light-emitting stylet with a light-detector connector extending from the light detector. The connecting step includes piercing the sterile drape with a piercing element of the drape-piercing connector before inserting the piercing element into a receptacle of the light-detector connector.

In some embodiments, the catheter is a central venous catheter (“CVC”). The advancing step includes advancing the CVC with the light-emitting stylet disposed therein through a right internal jugular vein, a right brachiocephalic vein, and into a superior vena cava (“SVC”).

In some embodiments, the catheter is a peripherally inserted central catheter (“PICC”). The advancing step includes advancing the PICC with the light-emitting stylet disposed therein through a right basilic vein, a right axillary vein, a right subclavian vein, a right brachiocephalic vein, and into an SVC.

In some embodiments, the method also includes a ceasing step of ceasing to advance the catheter through the vasculature of the patient after determining the distal-end portion of light-emitting stylet is located at the destination by way of the display screen.

These and other features of the concepts provided herein will become more apparent to those of skill in the art in view of the accompanying drawings and following description, which describe particular embodiments of such concepts in greater detail.

### DRAWINGS

FIG. 1 provides a block diagram of a first optical tip-tracking system in accordance with some embodiments.

FIG. 2 provides a block diagram of a second optical tip-tracking system in accordance with some embodiments.

FIG. 3 illustrates the first optical tip-tracking system in accordance with some embodiments.

FIG. 4 illustrates the first optical tip-tracking system including a catheter in accordance with some embodiments.

FIG. 5A illustrates a distal-end portion of a first light-emitting stylet of the first or second optical tip-tracking system in accordance with some embodiments.

FIG. 5B illustrates a transverse cross section of the first light-emitting stylet disposed in the catheter in accordance with some embodiments.

FIG. 6A illustrates a distal-end portion of a second light-emitting stylet of the first or second optical tip-tracking system in accordance with some embodiments.

FIG. 6B illustrates a transverse cross section of the second light-emitting stylet disposed in the catheter in accordance with some embodiments.

FIG. 7 illustrates a side view of a light detector of the first or second optical tip-tracking system in accordance with some embodiments.

FIG. 8 illustrate the first optical tip-tracking system in use in accordance with some embodiments.

FIG. 9 illustrate the second optical tip-tracking system in use in accordance with some embodiments.

FIG. 10 provides a transmission curve for optical window for biological tissue in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

Again, fluoroscopic methods typically used for tracking tips of medical devices such as guidewires and catheters expose clinicians and patients alike to harmful X-ray radiation. Magnetic and electromagnetic means for tracking the tips of the medical devices obviate some of the foregoing issues with respect to exposure to radiation, but the magnetic and electromagnetic means for tracking the tips of the medical devices are prone to interference.

Disclosed herein are optical tip-tracking systems and methods thereof that address the foregoing.

### Optical Tip-Tracking Systems

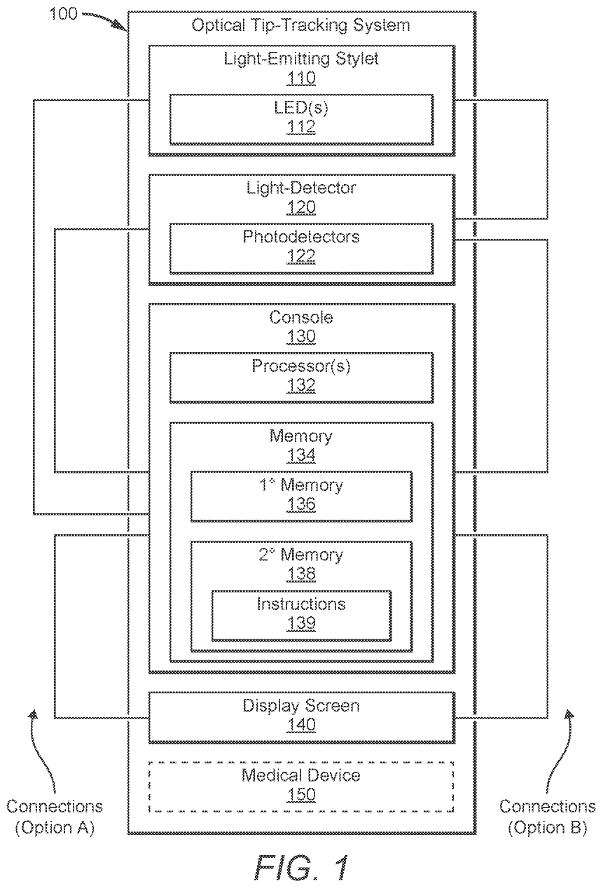

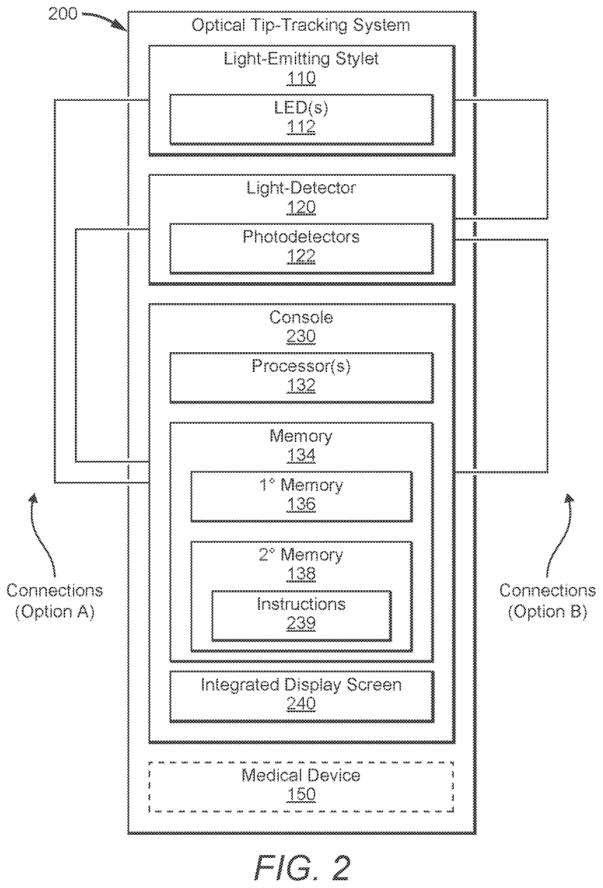

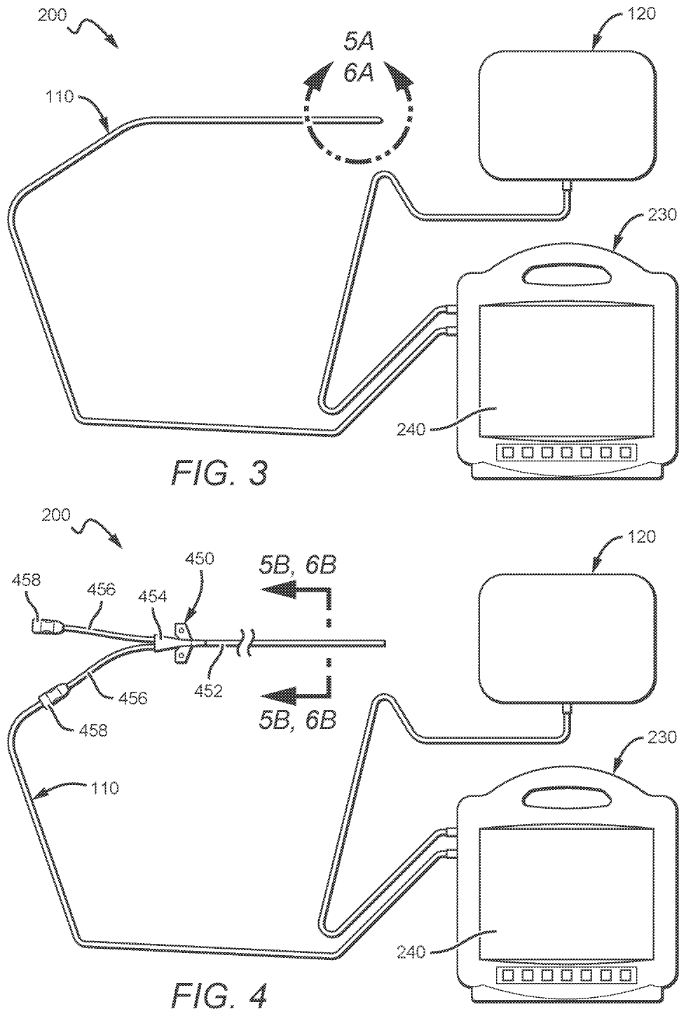

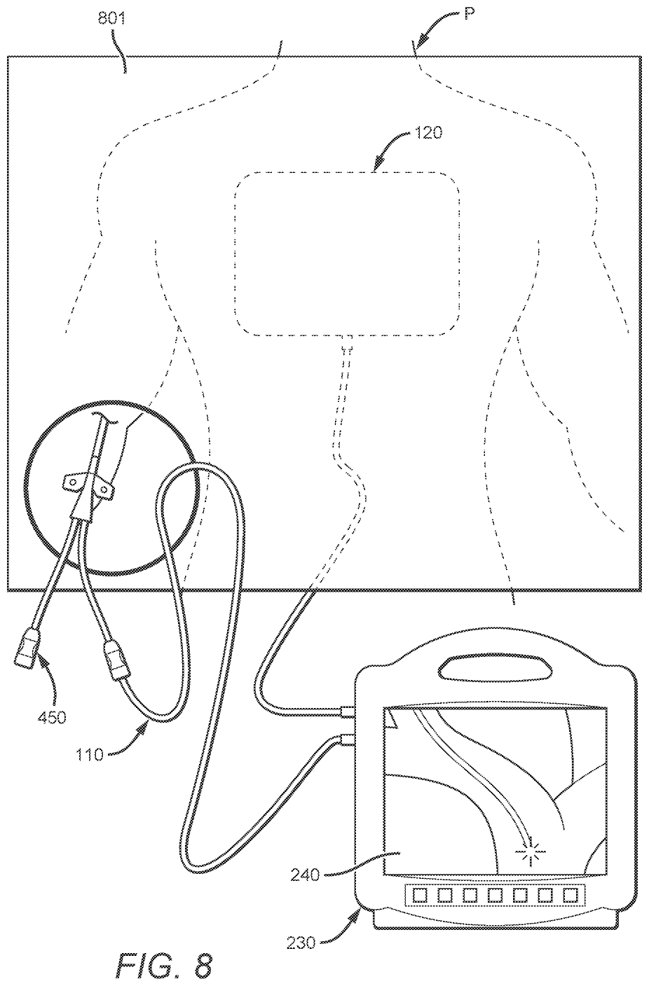

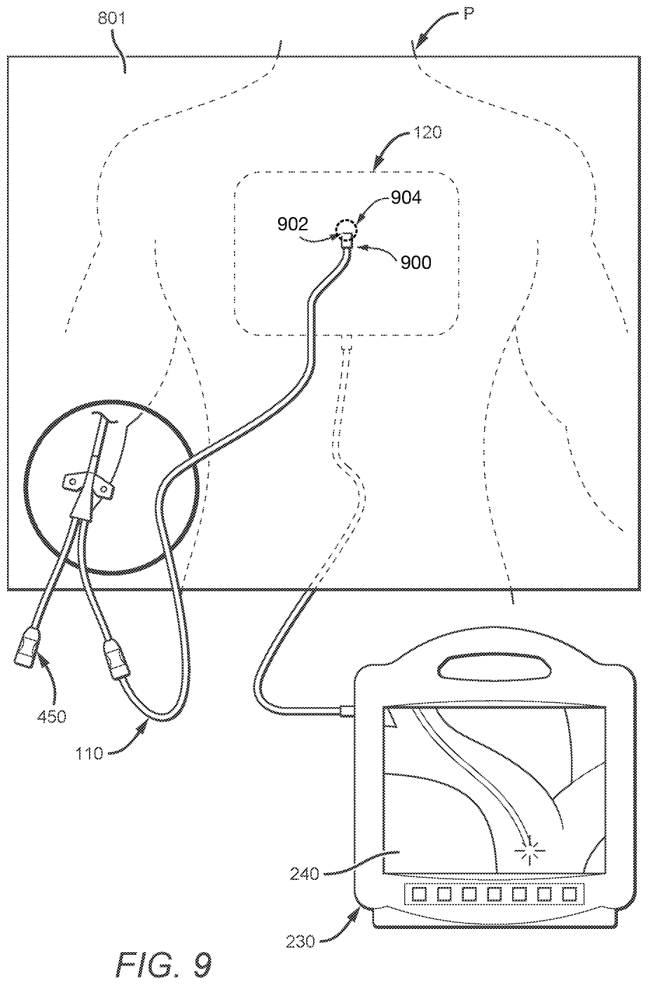

FIG. 1 provides a block diagram of a first optical tip-tracking system 100, FIG. 3 illustrates the first optical tip-tracking system 100, FIG. 4 illustrates the first optical tip-tracking system 100 including a catheter 450, and FIG. 8 illustrates the first optical tip-tracking system 100 in use, in accordance with some embodiments. FIG. 2 provides a block diagram of a second optical tip-tracking system 200 and FIG. 9 illustrates the second optical tip-tracking system 200 in use, in accordance with some embodiments.

As shown, the optical tip-tracking system 100 or 200 includes a light-emitting stylet 110, a light detector 120, and a console 130 or 230 configured to operably connect to the light-emitting stylet 110 and the light detector 120. Each optical tip-tracking system of the optical tip-tracking systems 100 and 200 also includes a display screen; however, the optical tip-tracking system 100 includes a standalone display screen 140, whereas the optical tip-tracking system 200 includes an integrated display screen 240. Each optical tip-tracking system of the optical tip-tracking systems 100 and 200 can also include a medical device 150 such as the catheter 450 of FIG. 4.

Beginning with the consoles 130 and 230, the console 130 or 230 includes memory 134 such as primary memory 136 and secondary memory 138. The primary memory 136 includes random-access memory (“RAM”). The secondary memory 138 includes non-volatile memory such as read-only memory (“ROM”) including a set of instructions 139 or 239 for loading into the primary memory 136 at runtime of the console 130 or 230.

One or more processors 132 are configured to instantiate an optical tip-tracking process in accordance with the instructions 139 or 239 for optically tracking a distal-end portion of the light-emitting stylet 110 while the light-emitting stylet 110 is disposed in a vasculature of a patient, a light source such as one or more LEDs 112 is emitting light, the light detector 120 is disposed over the light-emitting stylet 110, and a plurality of photodetectors of the light detector 120 such as photodetectors 122 are detecting the light emitted from the light source of the light-emitting stylet 110. The optical tip-tracking process is configured to provide tracking information as input to a display server of the console 130 or 230 for optically tracking the distal-end portion of the light-emitting stylet 110 in a graphical user interface on the display screen 140 or 240. Optically tracking the distal-end portion of the light-emitting stylet 110 can be animated on the display screen 140 or 240 as shown on the display screen 240 of the console 230 in FIGS. 8 and 9.

FIGS. 1 and 2 provide different connection options for connecting at least the light-emitting stylet 110 and the light detector 120 to the console 130 or 230.

The left-hand side of each figure or FIGS. 1 and 2 shows a connection option (“option A”) in which the light-emitting stylet 110 and the light detector 120 are independently connected to the console 130 or 230. For example, the console 130 or 230 and the light-emitting stylet 110 can be mutually configured such that the light-emitting stylet 110 directly connects to the console 130 or 230 or indirectly connects to the console 130 or 230 through an intervening multi-use cable having requisite conveying means for conveying electrical power (e.g., electrical leads), light (e.g., optical fiber), etc. Likewise, the console 130 or 230 and the light detector 120 can be mutually configured such that the light detector 120 directly connects to the console 130 or 230 or indirectly connects to the console 130 or 230 through an intervening multi-use cable.

The right-hand side of each figure of FIGS. 1 and 2 shows a connection option (“option B”) in which the light-emitting stylet 110 is connected to the light detector 120 and, in turn, the light detector 120 is connected to the console 130 or 230. A cable such as the intervening multi-use cable set forth above for connecting the light detector 120 to the console 130 or 230 includes the requisite conveying means for conveying electrical power (e.g., electrical leads), light (e.g., optical fiber), etc. The foregoing connection option is useful in that the light detector 120 is multi-use equipment configured to be placed over a patient P and under a sterile drape 801 outside a sterile field as shown in FIGS. 8 and 9. The light-emitting stylet 110 is single-use equipment intended for use within the foregoing sterile field. As shown in FIG. 9, the light-emitting stylet 110 can include a drape-piercing connector 900 in a proximal-end portion thereof having a piercing element 902 configured to both pierce the sterile drape 801 and insert into a receptacle 904 of a light-detector connector extending from the light detector 120 under the sterile drape 801. So configured, the intervening multi-use cable set forth above through which the light-emitting stylet 110 can be indirectly connected to the console 130 or 230 is not needed. For examples of drape-piercing connectors for the light-emitting stylet 110 and light-detector connectors for the light-detector 120 see U.S. Pat. No. 10,231,753 and children patents or patent applications thereof, each of which patents and patent application are incorporated herein by reference.

Both connection options of FIG. 1 show the display screen 140 connected to the console 130 in the optical tip-tracking system 100. FIG. 2 does not show connection options for connecting the integrated display screen 240 to the console 230 in the optical tip-tracking system 200. This is because the integrated display screen 240 is integrated into the console 240 as shown in FIGS. 3, 4, 8, and 9.

In addition to FIGS. 1-4, FIG. 7 illustrates a side view of the light detector 120 in accordance with some embodiments.

As set forth above, the light detector 120 is configured to be placed over a patient and under a sterile drape such as the sterile drape 801 of FIGS. 8 and 9.

The light detector 120 includes the photodetectors 122 disposed within the light detector 120 configured to detect the light emitted from the light source of the light-emitting stylet 110. The photodetectors 122 are arranged in an array such that the light emitted from the light source of the light-emitting stylet 110 remains detectable by at least one photodetector of the photodetectors 122 even when the light emitted from the light source of the light-emitting stylet 110 is anatomically blocked (e.g. by a rib) from another one or more photodetectors of the photodetectors 122.

The light detector 120 includes a housing having a patient-facing portion 722 of the housing configured to transmit at least a portion of the light emitted from the light source of the light-emitting stylet 110 to the photodetectors 122 disposed within the light detector 120. The housing also has a light-blocking portion 724 of the housing opposite the patient-facing portion 722 of the housing configured to block ambient light from the photodetectors 122 disposed within the light detector 120. In addition to the light-blocking portion 724 of the housing of the light detector 120, the sterile drape 801 also protects the photodetectors 122 from the ambient light while in use.

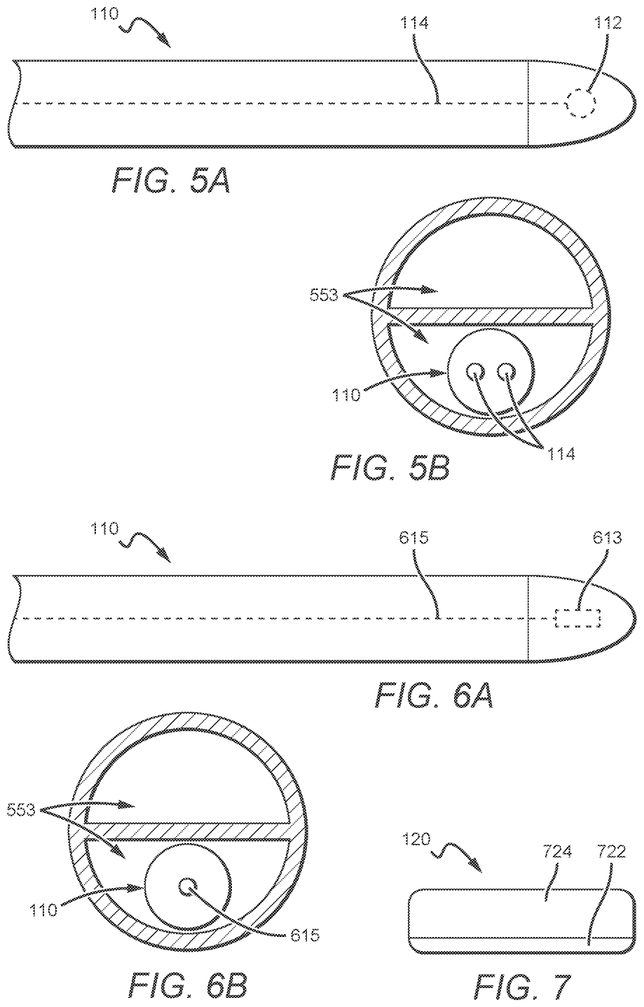

In addition to FIGS. 1-4, FIG. 5A illustrates the distal-end portion of the light-emitting stylet 110 and FIG. 5B illustrates a transverse cross section of the light-emitting stylet 110 in accordance with some embodiments having the one-or-more LEDs 112 as the light source. FIG. 6A illustrates the distal-end portion of the light-emitting stylet 110 and FIG. 6B illustrates a transverse cross section of the light-emitting stylet 110 in accordance with some embodiments having an external light source.

As set forth in more detail below, the light-emitting stylet 110 is configured to be disposed in a lumen of a catheter.

With respect to the light-emitting stylet 110 having the one-or-more LEDs 112 as the light source, the light-emitting stylet 110 includes the one-or-more LEDs 112 in a distal-end portion (e.g., a tip) of the light-emitting stylet 110 configured to emit light. The light-emitting stylet 110 also includes at least a pair of electrical leads 114 configured to convey electrical power from the console 130 or 230 to power the one-or-more LEDs 112. The distal-end portion of the light-emitting stylet 110 can be configured to directionally emit light from the one-or-more LEDs 112 in one or more chosen directions such as straight ahead in line with the light-emitting stylet 110, radially outward such as toward an extracorporeal surface of a patient, or a combination thereof.

With respect to the light-emitting stylet 110 having the external light source, the light-emitting stylet 110 includes an optical fiber 615 configured to convey light from the external source, for example, a light within the console 130 or 230, to the distal-end portion (e.g., the tip) of the light-emitting stylet 110 to emit light. A ferrule 613 disposed over a distal-end portion of the optical fiber 615 can be configured to directionally emit light from the optical fiber 615 in one or more chosen directions such as straight ahead in line with the light-emitting stylet 110, radially outward such as toward an extracorporeal surface of a patient, or a combination thereof.

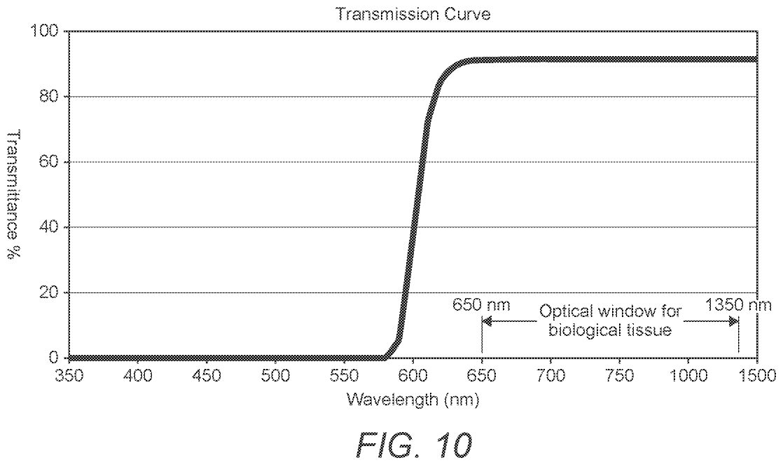

The light emitted from the light source can have a center wavelength between about 650 nm to 1350 nm including a center wavelength between about 650 nm to 950 nm or a center wavelength between about 1100 nm to 1350 nm. The light in the foregoing ranges of wavelengths is within an optical window for biological tissue in that such light penetrates biological tissue more deeply than light outside the foregoing ranges of wavelengths. (See FIG. 10 for the optical window for biological tissue.) Each LED of the one-or-more LEDs 112 can include aluminum gallium arsenide (AlGaAs) or gallium arsenide (GaAs) as a semiconductor material. In accordance with the semiconductor material and its composition, each LED of the one-or-more LEDs 112 can be configured to emit light at or within the following wavelengths: 660 nm, 680 nm, 800-850 nm, 850-940 nm, and 940 nm.

Adverting to FIG. 4, the catheter 450 can be a peripherally inserted central catheter (“PICC”), as shown, or a central venous catheter (“CVC”). In an example of a diluminal catheter, the catheter 450 can include a catheter tube 452, a bifurcated hub 454, two extension legs 456, and two Luer connectors 458 operably connected in the foregoing order. Notwithstanding the foregoing example, the catheter 450 can alternatively be a monoluminal catheter or a multi-luminal catheter including three or more lumens.

Continuing with the example of the diluminal catheter, the catheter 450 includes two lumens extending therethrough formed of adjoining lumen portions. Indeed, the catheter tube 452 includes two catheter-tube lumens 553. (See FIGS. 5B and 6B for the two catheter-tube lumens 553). The bifurcated hub 454 has two hub lumens correspondingly fluidly connected to the two catheter-tube lumens 553. Each extension leg of the two extension legs 456 has an extension-leg lumen fluidly connected to a hub lumen of the two hub lumens. Either lumen extending through the catheter 450 can accommodate the light-emitting stylet 110 disposed therein.

### Methods

A method of the optical tip-tracking system 100 or 200 includes a disposing step of disposing the light-emitting stylet 110 of the optical tip-tracking system 100 or 200 in a lumen of the catheter 450.

The method also includes a placing step of placing the light detector 120 of the optical tip-tracking system 100 or 200 over the patient P as shown in FIGS. 8 and 9.

The method also includes a placing step of placing the sterile drape 801 over both the patient P and the light detector 120.

As shown in FIG. 8, the method can also include a connecting step of connecting the drape-piercing connector of the light-emitting stylet 110 with the light-detector connector extending from the light detector 120. Such a connecting step includes piercing the sterile drape 801 with the piercing element of the drape-piercing connector before inserting the piercing element into the receptacle of the light-detector connector.

The method also includes an advancing step of advancing the catheter 450 from an insertion site to a destination within a vasculature of the patient P while emitting light from the light source (e.g., the one-or-more LEDs 112) and detecting the light with the photodetectors 122. The light source of the light-emitting stylet 110 should distally extends beyond a distal end of the catheter 450 while advancing the catheter 450, thereby enabling the photodetectors 122 of the light detector 120 to detect the light emitted from the light source of the light-emitting stylet 110.

When the catheter 450 is a CVC, the advancing step includes advancing the CVC with the light-emitting stylet 110 disposed therein through a right internal jugular vein, a right brachiocephalic vein, and into an SVC.

When the catheter 450 is a PICC, the advancing step includes advancing the PICC with the light-emitting stylet 110 disposed therein through a right basilic vein, a right axillary vein, a right subclavian vein, a right brachiocephalic vein, and into an SVC.

The method also includes a viewing step of viewing the display screen 140 or 240 of the optical tip-tracking system 100 or 200 while the display screen 140 or 240 graphically tracks the distal-end portion of the light-emitting stylet 110 through the vasculature of the patient P.

The method also includes a ceasing step of ceasing to advance the catheter 450 through the vasculature of the patient P after determining the distal-end portion of the light-emitting stylet 110 is located at the destination by way of the display screen 140 or 240.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A medical device tip-tracking system, comprising:
a light-emitting stylet configured to be disposed in a lumen of a medical device, the light-emitting stylet configured to emit light from a distal-end portion of the light-emitting stylet;
a light detector configured to be placed over a patient, the light detector including a plurality of photodetectors configured to detect the light emitted from the light source; and
a console coupled to the light-emitting stylet and the light detector, the console including:
a processor, and
non-volatile memory storing instructions that, when executed by the processor, cause performance of operations including display tracking information in a graphical user interface on a display screen as the light-emitting stylet is advanced through a vasculature of a patient.

2. The medical device tip-tracking system of claim 1, wherein the light-emitting stylet is configured to directly connect to the light detector.

3. The medical device tip-tracking system of claim 1, wherein the light-emitting stylet is configured to directly connect to the console.

4. The medical device tip-tracking system of claim 1, wherein the light-emitting stylet includes a light source at the distal-end portion, wherein the light source is configured to emit the light.

5. The medical device tip-tracking system of claim 4, wherein the light-emitting stylet is configured to indirectly connect to the console through the light detector such that electrical power passes from either the console to the light source or the light detector.

6. The medical device tip-tracking system of claim 1, wherein the light-emitting stylet includes an electrical lead configured to convey electrical power to the light source.

7. The medical device tip-tracking system of claim 1, wherein the light-emitting stylet includes an optical fiber configured to convey the light along a length of the light-emitting stylet, wherein the light is emitted from a distal-end portion of the optical fiber.

8. The medical device tip-tracking system of claim 7, wherein the light-emitting stylet includes a ferrule is disposed over a distal-end portion of the optical fiber, and wherein the ferrule is configured to direct the light in one or more directions.

9. The medical device tip-tracking system of claim 1, wherein the instructions, when executed by the processor, cause performance of further operations including:
graphically tracking the distal-end portion of the light-emitting stylet through the vasculature of the patient.

10. The medical device tip-tracking system of claim 1, wherein the console further includes the display screen integrated into a housing of the console.

11. A method of deploying an optical tip-tracking system, comprising:
disposing a light-emitting stylet in a lumen of a medical device, the light-emitting stylet configured to emit light from a distal-end portion of the light-emitting stylet;
placing a light detector over a patient, the light detector including a plurality of photodetectors configured to detect the light emitted from the light source; and
advancing the light-emitting stylet through a vasculature of a patient; and
viewing tracking information on a graphical user interface on a display screen as the light-emitting stylet is advanced through a vasculature of a patient.

12. The method of claim 11, wherein the light-emitting stylet is configured to directly connect to the light detector.

13. The method of claim 11, wherein the light-emitting stylet is configured to directly connect to the console.

14. The method of claim 11, wherein the light-emitting stylet includes a light source at the distal-end portion, wherein the light source is configured to emit the light.

15. The method of claim 14, wherein the light-emitting stylet is configured to indirectly connect to the console through the light detector such that electrical power passes from either the console to the light source or the light detector.

16. The method of claim 11, wherein the light-emitting stylet includes an electrical lead configured to convey electrical power to the light source.

17. The method of claim 11, wherein the light-emitting stylet includes an optical fiber configured to convey the light along a length of the light-emitting stylet, wherein the light is emitted from a distal-end portion of the optical fiber.

18. The method of claim 17, wherein the light-emitting stylet includes a ferrule is disposed over a distal-end portion of the optical fiber, and wherein the ferrule is configured to direct the light in one or more directions.

19. The method of claim 11, wherein the instructions, when executed by the processor, cause performance of further operations including:
graphically tracking the distal-end portion of the light-emitting stylet through the vasculature of the patient.

20. The method of claim 11, wherein the console further includes the display screen integrated into a housing of the console.
